(19) United States
(12) Patent Application Publication
(10) Pub. No.: US 2003/0203943 A1
(43) Pub. Date: Oct. 30, 2003

(54) Chemical complex comprising a pyridine carboxy derivative and an H2 histamine receptor antagonist

(75) Inventor: Morten Sloth Weidner, Virum (DK)

Correspondence Address: BIRCH STEWART KOLASCH & BIRCH, PO BOX 747, FALLS CHURCH, VA, 22040-0747, US

(73) Assignee: Astion Development Aps.

(21) Appl. No.: 10/430,507

(22) Filed: May 5, 2003

Related U.S. Application Data
(63) Continuation of application No. 09/813,719, filed on Mar. 21, 2001, now abandoned.
(60) Provisional application No. 60/191,690, filed on Mar. 23, 2000.

(30) Foreign Application Priority Data
Mar. 21, 2000 (DK) PA2000 00467

(51) Int. Cl. A61K 31/4433; A61K 31/455; C 07D 4 5/02

(52) U.S. Cl. 514/355; 546/283.4; 546/315

(57) ABSTRACT

The present invention relates to a chemical composition comprising a pyridine carboxy derivative and an H2 histamine receptor antagonist and a pharmaceutical composition or a dietary supplement comprising a pyridine carboxy derivative and an H2 histamine receptor antagonist and to the use of such compositions for the preparation of a medicament or a dietary-supplement for immunomodulation in a mammal and the suppression of hypersensitivity and/or inflammatory reaction.

### FIELD OF THE INVENTION

The present invention relates to a chemical composition comprising a pyridine carboxy derivative and an H2 histamine receptor antagonist and a pharmaceutical composition or a dietary supplement comprising a pyridine carboxy derivative and an H2 histamine receptor antagonist and to the use of such compositions for the preparation of a medicament or a dietary supplement for immunomodulation in a mammal and the suppression of hypersensitivity and/or inflammatory reaction.

### BACKGROUND OF THE INVENTION

Hypersensitivity is defined as a state of altered reactivity in which the body reacts with an exaggerated immune response to a substance (antigen). Hypersensitivity may be caused by exogenous or endogenous antigens.

Hypersensitivity reactions underlie a large number of diseases. Among these, allergic and autoimmune conditions are of great importance. A classification of hypersensitivity diseases is given in the textbook Clinical Medicine (Kumar, P. and Clark, M.: “Clinical Medicine”, 3rd edition, p. 147-150, 1994, Bailliere Tindall, London).

Type I hypersensitivity reactions (IgE mediated allergic reactions) are caused by allergens (specific exogenous antigens), e.g. pollen, house dust, animal dandruff, moulds, etc. Allergic diseases in which type I reactions play a significant role include asthma, eczema (atopic dermatitis), urticaria, allergic rhinitis and anaphylaxis.

Type II hypersensitivity reactions are caused by cell surface or tissue bound antibodies (IgG and IgM) and play a significant role in the pathogenesis of myasthenia gravis, Goodpasture's syndrome and Addisonian pernicious anaemia.

Type III hypersensitivity reactions (immune complex) are caused by autoantigens or exogenous antigens, such as certain bacteria, fungi and parasites. Diseases in which type III hypersensitivity reactions play a significant role include lupus erythematosus, rheumatoid arthritis and glomerulonephritis.

Type IV hypersensitivity reactions (delayed) are caused by cell or tissue bound antigens. This type of hypersensitivity plays a significant role in a number of conditions, e.g. graft-versus-host disease, leprosy, contact dermatitis and reactions due to insect bites.

A number of drug classes are available for the treatment of hypersensitivity reactions.

Among these the corticosteroids are some of the most widely used drugs. Corticosteroids primarily exert their pharmacological action by non-selectively inhibiting the function and proliferation of different classes of immune cells resulting in suppression of hypersensitivity reactions. Unfortunately, the corticosteroids are associated with a number of serious side effects, e.g. immuno-suppression, osteoporosis and skin atrophy.

Poly(ADP-ribose)polymerase, also known as poly(ADP-ribose)synthetase or poly(ADP-ribose)transferase is an nuclear enzyme that catalyses the posttranslational modification of nuclear proteins by covalent attachment of ADP-ribosyl moieties derived from NAD⁺ with an accompanying release of nicotinic acid amide. Preferred acceptor proteins are nuclear histones, whose poly-ADP-ribosylation induces local alterations in the architecture of chromatin domains.

Inhibitors of poly(ADP-ribose)polymerase have been found to suppress hypersensitivity reactions and inflammation.

Niacinamide, which is also known as nicotinamide, has been found to be a potent inhibitor of poly(ADP-ribose)polymerase.

Histamine is a biologically active amine that is found in many tissues, has complex pathological effects and is often released locally. There are several subclasses of histamine receptors, which are present in various tissues.

Binding of histamine to H2 receptors in the stomach plays a central role in relation to gastric acid secretion. Therefore histamine H2 receptor antagonists have been developed to reduce gastric acid secretion in relation to stomach ulcers.

Also cells of the immune system have H2 receptors through which histamine exerts immunomodulating effects.

The clinically most important histamin H2 receptor antagonists are ranitidine, cimetidine, famotidine and nizatidine.

Cancer is caused by an uncontrolled proliferation of cells that express varying degrees of fidelity to their precursors. These cancer cells form a malignant tumour that enlarges and may spread to adjacent tissues or through blood and lymph systems to other parts of the body. There are numerous forms of cancer of varying severity. For most types of cancer there is no effective treatment today.

### SUMMARY OF THE INVENTION

It has been found by the present inventor that a chemical complex or a pharmaceutical composition comprising a pyridine carboxy derivative and an H2 histamine receptor antagonist and optionally a pharmaceutically acceptable carrier significantly suppresses hypersensitivity reactions.

Compared to existing therapeutic agents, such as corticosteroids or non-steroidal anti-inflammatory drugs, the chemical complexes and pharmaceutical compositions according to the present invention have the advantage of not being likely to be associated with any serious side effects, as all of their components are non-toxic and well tolerated by the organism in the pharmacologically relevant doses.

Due to the pharmacological effects mentioned above, the chemical complexes and pharmaceutical compositions according to the invention can be employed for the following therapeutic applications:

Immunomodulation.

Treatment or prevention of hypersensitivity diseases.

Treatment or prevention of IgE mediated allergic reactions and conditions.

Treatment or prevention of autoimmune disorders.

Alleviation of pain.

Treatment or prevention of cancer.

Accordingly, the present invention provides a chemical complex or a pharmaceutical composition comprising:

i) an optionally substituted pyridine carboxy derivative according to formula 1

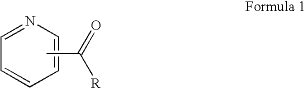

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

ii) an H2 histamine receptor antagonist; and optionally

iii) a pharmaceutically acceptable carrier.

Furthermore, the present invention provides the use of a chemical complex or a composition comprising a pyridine carboxy derivative and an H2 histamine receptor antagonist as described above and optionally a pharmaceutically acceptable carrier for the preparation of a medicament for immunomodulation in a mammal, for the suppression of hypersensitivity reactions in a mammal, such as IgE mediated allergic reactions, and autoimmune reactions in a mammal, and for the alleviation of pain in a mammal, the mammal preferentially being a human.

Thus, according to the invention a chemical complex or a composition comprising a pyridine carboxy derivative and an H2 histamine receptor antagonist as described above and optionally a pharmaceutically acceptable carrier can be used in a method for the treatment or prevention of a hypersensitivity disease in a mammal, said method comprising administering said chemical complex or said composition to said mammal; and the invention comprises the use of said chemical complex or said composition for the preparation of a medicament for the treatment or prevention of hypersensitivity diseases in a mammal.

Also, according to the invention a chemical complex or a composition comprising a pyridine carboxy derivative and an H2 histamine receptor antagonist as described above and optionally a pharmaceutically acceptable carrier can be used in a method for the treatment or prevention of an autoimmune disorder in a mammal, said method comprising administering said chemical complex or said composition to said mammal; and the invention comprises the use of said chemical complex or said composition for the preparation of a medicament for the treatment or prevention of autoimmune disorders in a mammal.

Further, according to the invention a chemical complex or a composition comprising a pyridine carboxy derivative and an H2 histamine receptor antagonist as described above and optionally a pharmaceutically acceptable carrier can be used in a method for the treatment or prevention of an IgE mediated allergic reaction or condition in a mammal, said method comprising administering said chemical complex or said composition to said mammal; and the invention comprises the use of said chemical complex or said composition for the preparation of a medicament for the treatment or prevention of IgE mediated allergic reactions and conditions in a mammal.

Also, according to the invention a chemical complex or a composition comprising a pyridine carboxy derivative, an H2 histamine receptor antagonist as described above and optionally a pharmaceutically acceptable carrier can be used in a method for the alleviation of pain in a mammal, said method comprising administering said chemical complex or said composition to said mammal; and the invention comprises the use of said chemical complex or said composition for the preparation of a medicament for the alleviation of pain in a mammal.

Further, according to the invention a chemical complex or a composition comprising a pyridine carboxy derivative, an H2 histamine receptor antagonist as described above and optionally a pharmaceutically acceptable carrier can be used in a method for the treatment or prevention of cancer in a mammal, said method comprising administering said chemical complex or said composition to said mammal; and the invention comprises the use of said chemical complex or said composition for the preparation of a medicament for the treatment or prevention of cancer in a mammal.

### DETAILED DESCRIPTION OF THE INVENTION

It has been found by the present inventor that a chemical complex or a composition comprising a pyridine carboxy derivative, said pyridine carboxy derivative preferably being selected from the group consisting of niacinamide and derivatives thereof; an H2 histamine receptor antagonist, said H2 histamine receptor antagonist preferably being selected from the group consisting of ranitidine, cimetidine, famotidine, nizatidine and derivatives thereof; and optionally a pharmaceutically acceptable carrier significantly suppresses hypersensitivity reactions.

Such chemical complexes or compositions are novel and provide a surprisingly good anti-hypersensitivity and anti-inflammatory effect with a surprisingly good safety profile. Thus the chemical complexes or compositions of the invention are virtually non-toxic and yet very therapeutically effective. The present inventor puts forward the hypothesis that the very beneficial therapeutic index of the compositions of the invention compared to single chemical anti-hypersensitivity drugs is due to the more complex nature of the compositions of the invention, giving a lower toxic load on the body of any single chemical compound and yet giving a surprisingly good therapeutic effect, due to synergistic effects between the components of the compositions.

More specifically, the above mentioned chemical complexes or compositions of the invention provide the following pharmacological effects upon administration to the living organism:

Immunomodulation;

Suppression of hypersensitivity reactions;

Suppression of IgE mediated allergic reactions;

Suppression of autoimmune reactions;

Reduction of pain;

Treatment or prevention of cancer;

Accordingly, the present invention provides a chemical complex or a pharmaceutical composition comprising:

i) an optionally substituted pyridine carboxy derivative according to formula 1

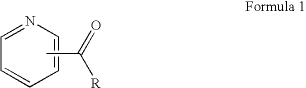

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

ii) an H2 histamine receptor antagonist; and optionally

iii) a pharmaceutically acceptable carrier, wherein

the ratio between the inhibitor of poly(ADP-ribose)polymerase and the H2 histamine receptor antagonist is in the range from 1:1000 to 1000:1, e.g. from 1:100 to 100:1, such as from 1:50 to 50:1, such as from 1:40 to 40:1, preferably from 1:30 to 30:1, e.g. from 1:25 to 25:1, such as from 1:20 to 20:1, more preferably in the range from 1:18 to 18:1, e.g. from 1:16 to 16:1, such as from 1:14 to 14:1, e.g. from 1:12 to 1:12, more preferably from 1:10 to 10: 1, such as from 1:9 to 9: 1, e.g. from 1:8 to 8: 1, such as from 1:7 to 7:1, e.g. from 1:6 to 6:1, most preferably from 1:5 to 5:1, such as from 1:4 to 4:1, e.g. from 1:3 to 3:1, such as from 1:2 to 2:1.

According to the invention an H2 histamine receptor antagonist is defined as any competitive or irreversible H2 histamine receptor antagonist. Thus according to the invention the antagonist or a prodrug thereof may be used as the H2 histamine receptor antagonist in the complexes or compositions of the invention. Non-limiting examples of such antagonists are ranitidine (e.g. in the form of ranitidine hydrochloride), cimetidine (e.g. in the form of cimetidine hydrochloride), famotidine, nizatidine and derivatives thereof. Accordingly such derivatives may be obtained through any kind of chemical modification of the H2 histamine receptor antagonists.

In a preferred embodiment of the invention the pyridine carboxy derivative is selected from the group consisting of niacinamide, nicotinic acid, methyl nicotinate, ethyl nicotinate, N2-methylniacinamide and N2-ethylniacinamide.

The term “optionally substituted” is intended to mean the substitution of one or more hydrogen atoms is substituted with another atom, chemical group or entity, termed substituents. Illustrative examples of substituents include carboxyl, formyl, amino, hydroxyl, halogen, nitro, sulphono, sulphanyl, C₁₋₆-alkyl, aryl, aryloxy, aryloxycarbonyl, arylcarbonyl, heteroaryl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, C₁₋₆-alkoxyl, oxo, C₁₋₆-carboxyl, C₁₋₆-alkoxycarbonyl, C₁₋₆-alkylcarbonyl, where aryl and heteroaryl representing substituents may be substituted 1-5 times with C₁₋₆-alkyl, C₁₋₆-alkoxy, nitro, cyano, hydroxy, amino or halogen. In general, the above substituents may be susceptible to further optional substitution.

The term C₁-C₂₀ alkyl is intended to mean a linear or branched saturated hydrocarbon chain wherein the longest chains has from one to twenty carbon atoms, such as methyl, ethyl, n-propyl, isopropyl, n-butyl, isobutyl, sec-butyl, tert-butyl, pentyl, isopentyl, neopentyl, hexyl, heptyl, octyl, undecacyl, dodecyl, etc. A branched hydrocarbon chain is intended to mean a C₁₋₂₀-alkyl substituted at any carbon with a hydrocarbon chain. The C₁-C₂₀ alkyl chain of the present invention may be optionally substituted.

The term “halogen” includes fluorine, chlorine, bromine and iodine.

It should also be understood that salts of compounds of formula 1 are anticipated, including, for instance hydrates and solvent addition forms. The term “base addition salts” include alkali metals, such as sodium and potassium, alkali earth metals, such as calcium and magnesium, and organic addition salts such as quaternary ammonium cations.

The chemical complex of the present invention relates to a complex obtainable from the combining of a pyridine carboxy derivative of Formula 1 and a glucosaminoglycan or a fragment or derivative thereof.

As stated, the complex comprises, in part, the optionally substituted pyridine carboxy derivative according to Formula 1 wherein R may be selected from OH; OR′; NH₂; NHR′; NR′R″, O⁻Y⁺, and halogen. R′ and R″ may independently be selected from optionally substituted C₁-C₂₀ alkyl.

The optionally substituted pyridine carboxy derivative may, for illustrative purposes, be selected from the group consisting of optionally substituted nicotinic acid, its corresponding acyl halide, ester, acid salt, or amide, nicotinamide; optionally substituted isonicotinic acid, its corresponding acyl halide, ester, acid salt, or amide, isonicotinamide; and optionally substituted picolinic acid, its corresponding acyl halide, ester, acid salt, or amide, picolinamide.

In the embodiment where the optionally substituted pyridine carboxy derivative is an amide, the amide may be its free primary amide (NH₂), its secondary amide (NHR′) or its tertiary amide (NR′R″).

As stated, the pyridine carboxy derivative may be optionally substituted. In one suitable embodiment, the pyridine carboxy is further substituted with a carboxy group such as a carboxylic acid, acyl halide, carboxylic ester, or acetamide. The pyridine carboxy may be substituted 0 to 4 times, such as 0, 1, 2, 3, or 4 times, preferably 0 to 1 time, most preferably 0 times.

According to the invention the above-mentioned chemical complexes or compositions can be combined with any other active ingredient to potentiate the therapeutic action.

“A “dietary supplement” is defined according to the U.S. Food and Drug Administration in the Dietary Supplement Health and Education Act of 1994 (DSHEA).The DSHEA gives the following formal definition of a “dietary supplement”:

“A dietary supplement:

is a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these things.

is intended for ingestion in pill, capsule, tablet, or liquid form.”

Similar definitions exist in other parts of the world, e.g. in Europe; in the present context, the definition is as defined above. Different denominations concerning “dietary supplements” are used around the world, such as “food supplements”, “neutraceuticals”, “functional foods” or simply “foods”. In the present context the term “dietary supplement” covers any such denomination or definition.

“Systemic administration” is defined as administration by the parenteral route such as the intravenous, intraperitoneal, intraarticular, intraventricular, intracapsular, intraspinal, intramuscular, subcutaneous, intradermal, oral, buccal, sublingual, nasal, rectal, vaginal or transdermal routes.

“Topical administration” is used in its conventional sense to mean delivery of a topical chemical complex or pharmacologically active composition to the skin or mucosa.

The above mentioned pharmacological actions provide part of the rationale for the following therapeutic applications of a chemical complex or a composition comprising a pyridine carboxy derivative and an H2 histamine receptor antagonist and optionally a pharmaceutically acceptable carrier:

A method for the treatment or prevention of hypersensitivity disease or inflammation characterised by the administration of the above mentioned chemical complexes or compositions to a mammal, preferentially a human. The therapeutic action may be relevant to all known diseases associated with hypersensitivity reactions or inflammation. Autoimmune disorders and IgE mediated allergic conditions are described below in more detail. Besides these specific therapeutic areas, the action of the above mentioned composition is relevant to all known conditions and diseases associated with hypersensitivity reaction, and the following examples are not limiting with respect to this: infections (viral, bacterial, fungal, parasitic, etc.), cold and flu, contact dermatitis, insect bites, allergic vasculitis, postoperative reactions, transplantation rejection (graft-versus-host disease), etc.

A method for the treatment or prevention of autoimmune disorders characterised by the administration of the above mentioned chemical complexes or compositions to a mammal, preferentially a human. The applicant puts forward the hypothesis that the therapeutic action is due to the immunomodulating and suppressing effect on hypersensitivity reactions of the above mentioned chemical complex or composition. The therapeutic action may be relevant to all known autoimmune disorders and the following examples are not limiting with respect to this: Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjögren's Disease, Lupus Erythematosus, Primary vasculitis, Rheumatoid Arthritis, Juvenile Arthritis, Mixed Connective Tissue Disease, Psoriasis, Pemfigus, Pemfigoid, Dermatitis Herpetiformis, etc.

A method for the treatment or prevention of an IgE mediated allergic reaction or condition characterised by the administration of the above mentioned chemical complexes or compositions to a mammal, preferentially a human. The applicant puts forward the hypothesis that the therapeutic action is due to the suppressing effect on hypersensitivity reaction of the above mentioned compositions. The therapeutic action may be relevant to all known IgE mediated allergic reactions and conditions, and the following examples are not limiting with respect to this: asthma, eczema (e.g. atopic dermatitis), urticaria, allergic rhinitis, anaphylaxis, etc.

A method for the treatment or prevention of any condition associated with pain characterised by the administration of the above mentioned chemical complexes or compositions to a mammal, preferentially a human. The applicant puts forward the hypothesis that the therapeutic action is related to immunomodulation, possibly to a suppressing effect on hypersensitivity reactions.

Accordingly, the chemical complexes or compositions of the invention are suitable for the treatment or prevention of diseases caused by inflammation of various tissues, e.g. inflammation of the prostate, in particular prostatitis.

“Prostatitis” is defined as inflammatory conditions affecting the prostate, including acute and chronic infections with specific bacteria and, more commonly, instances in which signs and symptoms of prostatic inflammation are present but no specific organism can be detected.

Also, the chemical complexes or compositions of the invention may be employed for the treatment or prevention of cancer of any type and at any stage. The present inventor puts forward the hypothesis that the anticancer effect is due to a combination of immunomodulating and tumour-suppressing effects of the complexes and compositions of the invention.

According to the invention the above mentioned chemical complexes or compositions can be combined with any other active ingredients to potentiate the therapeutic action.

In a preferred embodiment of the invention the above mentioned chemical complexes or compositions are used for systemic administration.

In another preferred embodiment of the invention the above mentioned chemical complexes or compositions are used for topical administration.

A pharmaceutical acceptable carrier for systemic or topical administration can be water or vehicles other than water, said other vehicles can be used in the compositions and can include solids or liquids such as emollients, solvents, humectants, thickeners and powders. Examples of each of these types of vehicles, which can be used singly or as compositions of one or more vehicles, are as follows:

Emollients, such as stearyl alcohol, glyceryl monoricinoleate, glyceryl monostearate, propane-1,2-diol, butane-1,3-diol, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate;

solvents, such as water, methylene chloride, isopropanol, castor oil, ethylene glycol monoethyl ether, diethylene glycol monobutyl ether, diethylene glycol monoethyl ether, dimethyl sulfoxide, tetrahydrofuran, vegetable and animal oils, glycerol, ethanol, propanol, propylene glycol, and other glycols or alcohols, fixed oils;

humectants or moistening agents, such as glycerin, sorbitol, sodium 2-pyrrolidone-5-carboxylate, soluble collagen, dibutyl phthalate, gelatin;

powders, such as chalk, talc, kaolin, starch and derivatives thereof, gums, colloidal silicon dioxide, sodium polyacrylate, chemically modified magnesium aluminium silicate, hydrated aluminium silicate, carboxyvinyl polymer, sodium carboxymethyl cellulose, ethylene glycol monostearate;

gelling or swelling agents, such as pectin, gelatin and derivatives thereof, cellulose derivatives such as methyl cellulose, carboxymethyl cellulose or oxidised cellulose, cellulose gum, guar gum, acacia gum, karaya gum, tragacanth gum, bentonite, agar, alginates, carbomer, gelatine, bladderwrack, ceratonia, dextran and derivatives hereof, ghatti gum, hectorite, ispaghula husk, xanthan gum;

polymers, such as polylactic acid or polyglycolic acid polymers or copolymers thereof, paraffin, polyethylene, polyethylene oxide, polyethylene glycol, polypropylene glycol, polyvinylpyrrolidone;

surfactants, such as non-ionic surfactants, e.g. glycol and glycerol esters, macrogol ethers and esters, sugar ethers and esters, such as sorbitan esters, ionic surfactants, such as amine soaps, metallic soaps, sulfated fatty alcohols, alkyl ether sulfates, sulfated oils, and ampholytic surfactants and lecitins;

buffering agents, such as sodium, potassium, aluminium, magnesium or calcium salts (such as the chloride, carbonate, bicarbonate, citrate, gluconate, lactate, acetate, gluceptate or tartrate).

The active ingredients of the chemical complex or pharmaceutical composition of the present invention need not be administered as one pharmaceutically entity, but can of course be administered as individual compounds or pharmaceutical compositions, i.e. as

ia) an optionally substituted pyridine carboxy derivative according to formula 1

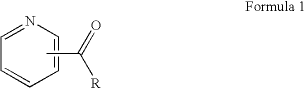

wherein R may be selected from OH; OR′; NH₂; NHR′; NR′R″, O⁻Y⁺, and halogen, wherein R′ and R″ may independently be selected from optionally substituted C₁-C₂₀ alkyl; and Y is a base addition salt of the free carboxylate; and

i) a pyridine carboxy derivative according to formula 1

optionally with iia) a pharmaceutically acceptable carrier

as one component as the one pharmaceutically entity, and

ib) an H2 histamine receptor antagonist;

and optionally iib) a pharmaceutically acceptable carrier

as the second pharmaceutically entity.

Furthermore, it is obvious that in the use according to the invention for the preparation of medicaments or dietary supplements, the above mentioned compositions may be mixed with additives such as surfactants, solvents, thickeners, stabilisers, preservatives, antioxidants, flavours, etc. to obtain a desirable product formulation suitable for systemic administration. Similarly, a pharmaceutical or dietary supplement according to the invention may further contain such additives. There are no limitations on the route of administration or dosage form of the formulation, and the following examples are not limiting with respect to this: tablets, capsules, lozenges, chewing gum, fluids, granulates, sprays (e.g. aerosol), inhalants, etc. Optionally, the composition may also contain surfactants such as bile salts, polyoxyethylene-sorbitan-fatty acid esters or polyalcohol mixed chain-length fatty acid esters for improving dispersibility of the composition in the digestive fluids leading to improved bioavailability or for obtaining the final dosage form of the composition.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver compositions of the invention. Additionally, the compositions may be delivered using a sustained-release system, such as semi-permeable matrices of solid polymers containing the therapeutic agent. Various sustained-release materials have been established and are well known by those skilled in the art. Sustained-release capsules may, depending on their chemical nature, release the compositions for a few weeks up to over 100 days.

Furthermore, the invention relates to a method for the preparation of a chemical complex or a pharmaceutically active composition as described above characterised by obtaining an H2 histamine receptor antagonist preferably being selected from the group consisting of ranitidine, cimetidine, famotidine, nizatidine and derivatives thereof, and a pyridine carboxy derivative according to formula 1 as described above; and mixing said H2 histamine receptor antagonist and pyridine carboxy derivative, optionally with a pharmaceutically acceptable carrier.

### EXAMPLES

### Example 1

A ranitidine hydrochloride/niacinamide complex 1:8 (mol/mol) is prepared:

1000 g ranitidine hydrochloride and 2783.7 g niacinamide is dissolved in as little water as possible. The mixture is spray dried to give a white powder.

### Example 2

A ranitidine hydrochloride/niacinamide complex 1:8 (mol/mol) is prepared:

1000 g ranitidine hydrochloride and 2783.7 g niacinamide is dissolved in as little water as possible. The mixture is freeze dried to give a white powder.

### Example 3

A ranitidine hydrochloride/niacinamide complex 1:16 (mol/mol) is prepared:

1000 g ranitidine hydrochloride and 5567.4 g niacinamide is dissolved in as little water as possible. The mixture is spray dried to give a white powder.

### Example 4

A ranitidine hydrochloride/niacinamide complex 1:16 (mol/mol) is prepared:

1000 g ranitidine hydrochloride and 5567.4 g niacinamide is dissolved in as little water as possible. The mixture is freeze dried to give a white powder.

### Example 5

A famotidine/niacinamide complex 1:8 (mol/mol) is prepared:

1000 g famotidine and 2894.2 g niacinamide is dissolved in as little water as possible.

The mixture is spray dried to give a white powder.

### Example 6

A famotidine/niacinamide complex 1:8 (mol/mol) is prepared:

1000 g famotidine and 2894.2 g niacinamide is dissolved in as little water as possible.

The mixture is freeze dried to give a white powder.

### Example 7

A famotidine/niacinamide complex 1:16 (mol/mol) is prepared:

1000 g famotidine and 5788.4 g niacinamide is dissolved in as little water as possible.

The mixture is spray dried to give a white powder.

### Example 8

A famotidine/niacinamide complex 1:16 (mol/mol) is prepared:

1000 g famotidine and 5788.4 g niacinamide is dissolved in as little water as possible.

The mixture is freeze dried to give a white powder.

### Example 9

A cimetidine/niacinamide complex 1:8 (mol/mol) is prepared:

1000 g cimetidine and 3871.6 g niacinamide is dissolved in as little 50% ethanol as possible. The mixture is freeze dried to give a white powder.

### Example 10

A cimetidine/niacinamide complex 1:16 (mol/mol) is prepared:

1000 g cimetidine and 7743.2 g niacinamide is dissolved in as little 50% ethanol as possible. The mixture is freeze dried to give a white powder.

### Example 11

A nizatidine/niacinamide complex 1:8 (mol/mol) is prepared:

1000 g nizatidine and 2947.0 g niacinamide is dissolved in as little 50% ethanol as possible. The mixture is freeze dried to give a white powder.

### Example 12

A nizatidine/niacinamide complex 1:16 (mol/mol) is prepared:

1000 g nizatidine and 5893.9 g niacinamide is dissolved in as little 50% ethanol as possible. The mixture is freeze dried to give a white powder.

### Example 13

A ranitidine hydrochloride/N2-methylniacinamide complex 1:8 (mol/mol) is prepared:

1000 g ranitidine hydrochloride and 3102.9 g N2-methylniacinamide is dissolved in as little water as possible. The mixture is spray dried to give a white powder.

### Example 14

A ranitidine hydrochloride/N2-methylniacinamide complex 1:16 (mol/mol) is prepared:

1000 g ranitidine hydrochloride and 6205.8 g N2-methylniacinamide is dissolved in as little water as possible. The mixture is spray dried to give a white powder.

### Example 15

A ranitidine hydrochloride/N2-ethylniacinamide complex 1:8 (mol/mol) is prepared:

1000 g ranitidine hydrochloride and 3422.1 g N2-ethylniacinamide is dissolved in as little 50% ethanol as possible. The mixture is freeze dried to give a white powder.

### Example 16

A ranitidine hydrochloride/N2-ethylniacinamide complex 1:16 (mol/mol) is prepared:

1000 g ranitidine hydrochloride and 6844.1 g N2-ethylniacinamide is dissolved in as little 50% ethanol as possible. The mixture is freeze dried to give a white powder.

### Example 17

A ranitidine hydrochloride/nicotinic acid complex 1:8 (mol/mol) is prepared:

1000 g ranitidine hydrochloride and 2806.5 g nicotinic acid is dissolved in as little water as possible. The mixture is spray dried to give a white powder.

### Example 18

A ranitidine hydrochloride/nicotinic acid complex 1:16 (mol/mol) is prepared:

1000 g ranitidine hydrochloride and 5613.0 g nicotinic acid is dissolved in as little water as possible. The mixture is spray dried to give a white powder.

### Example 19

A ranitidine hydrochloride/methylnicotinate complex 1:8 (mol/mol) is prepared:

1000 g ranitidine hydrochloride and 3125.7 g methylnicotinate acid is dissolved in as little 50% ethanol as possible. The mixture is freeze dried to give a white powder.

### Example 20

A ranitidine hydrochloride/methylnicotinate complex 1:16 (mol/mol) is prepared:

1000 g ranitidine hydrochloride and 6251.4 g methylnicotinate acid is dissolved in as little 50% ethanol as possible. The mixture is freeze dried to give a white powder.

### Example 21

A pharmaceutical composition according to the invention is prepared as follows:

A gelatine capsule containing a ranitidine hydrochloride/niacinamide complex 1:8 (mol/mol) is prepared:

1000 g ranitidine hydrochloride and 2783.7 g niacinamide is dissolved in as little water as possible. The mixture is spray dried to give a white powder. 500 mg of the powder is transferred to a hard gelatin capsule.

### Example 22

A pharmaceutical composition according to the invention is prepared as follows:

A gelatine capsule containing a ranitidine hydrochloride/niacinamide complex 1:16 (mol/mol) is prepared:

1000 g ranitidine hydrochloride and 5567.4 g niacinamide is dissolved in as little water as possible. The mixture is spray dried to give a white powder. 500 mg of the powder is transferred to a hard gelatin capsule.

### Example 23

A pharmaceutical composition according to the invention is prepared as follows:

A gelatine capsule containing 500 mg of a ranitidine hydrochloride/niacinamide complex 1:8 (mol/mol) is prepared:

132 mg ranitidine hydrochloride and 368 mg niacinamide is transferred to a hard gelatin capsule.

### Example 24

A pharmaceutical composition according to the invention is prepared as follows:

A gelatine capsule containing 500 mg of a ranitidine hydrochloride/niacinamide complex 1:16 (mol/mol) is prepared:

76 mg ranitidine hydrochloride and 424 mg niacinamide is transferred to a hard gelatin capsule.

### Example 25

A pharmaceutical composition according to the invention is prepared as follows:

A gelatine capsule containing a ranitidine hydrochloride/N2-ethylniacinamide complex 1:8 (mol/mol) is prepared:

1000 g ranitidine hydrochloride and 3422.1 g N2-ethylniacinamide is dissolved in as little 50% ethanol as possible. The mixture is freeze dried to give a white powder. 500 mg of the powder is transferred to a hard gelatin capsule.

### Example 26

A pharmaceutical composition according to the invention is prepared as follows:

A gelatine capsule containing 250 mg of a ranitidine hydrochloride/ethylnicotinate complex 1:8 (mol/mol) is prepared:

56 mg ranitidine hydrochloride and 194 mg methylnicotinate is transferred to a hard gelatin capsule.

### Example 27

Study Object

The immunomodulating and anti-inflammatory effects of complexes or compositions of the invention are tested in vitro. The model used is IL-β secretion in human peripheral blood mononuclear leukocytes. Dexamethasone is employed as positive control.

Test Compounds

Any of the complexes or compositions according to examples 1 to 20 are tested.

Cellular IL-β assay

The study is performed employing a modification of the methods of Page et al (Int. J. Oncology 3: 473-476, 1993) and Welker et al (Int. Arch. Of Allergy and Immunology, 109: 110-115, 1996). The test compound is dissolved in water or dimethylsulfoxide for the cellular assay. The test compounds are tested in duplicate at the following concentrations: 0.8 μg/ml, 4.0 μg/ml, 20.0 μg/ml, 100.0 μg/ml and 500.0 μg/ml.

The active compounds or DMSO 0.4% (control) are incubated with lipopolysaccharide-stimulated (25 ng/ml) human peripheral blood mononuclear leukocytes in growth medium RPMI-1640, pH 7.4 for 16 hours at 37° C. The IL-β cytokine levels in the conditioned medium are quantitated using a sandwich ELISA kit.

Findings and Interpretation

The complexes or compositions of the invention dose-dependently inhibit the secretion of IL-β . Similar tests may be employed where other pro-inflammatory cytokines are measures, e.g. TNF-α, IL-6 and IL-8. Similar tests may also be employed where pro-allergic cytokines are measures, e.g. IL-4, IL-5 or IL-13.

### Example 28

Study Object

The effects of complexes or compositions of the invention are tested on tumor progression in SCID mice xenografted with colorectal adenocarcinoma cells. The aim of the study is to produce growth curves of the grafted tumour and to monitor the effect of increasing doses of the complexes or compositions of the invention on the growth curves.

Test Compounds

Any of the complexes or compositions according to examples 1 to 20 are tested.

Dosing Pattern

The test compound is dissolved in water. The test compound is administered orally once daily for 3 consecutive days at 100, 300 and 1000 mg/kg. Dosing volume is 5 ml/kg.

Animals

In this study, female SCID mice with an age of 6 weeks are used. Ten mice are used per group. The mice are caged in standard polypropylene cages (l×w×h=40×25×15 cm). Bedding is Hahnflock S 8/15, produced by Hahn & Co., Faserstoffwerk, Bredenbeck-Kronsburg, Germany. Bedding is changed twice a week in a laminar flow unit. Cages are housed in Scantainers with HEPA-filters (Class EU10, withholding 98.5% of all particles>0.3 μm. The air is exchanged appr.70 times/hour in the Scantainer. Temperature is 18° C.-22° C., and is controlled via the ambient ventilation system in the laboratory. Light cycle is 12-hour dark and 12-hour light (lights on 06.00). Diet is Altromin 1314 special formulation, Produced by Altromin Denmark, Chr. Pedersen A/S, 4100 Ringsted, Denmark. Water is acidified with HCl, and is changed at least every third day. Diet and water is administered ad libitum.

Method

The mice are randomised to test groups of ten mice and the daily administration of test compound starts and continues until the termination of the study. After one week each mouse is injected subcutaneously With appr. 2.5×10⁶ cells contained in 0.1 ml of Hank's Balanced Salts Solution (HBSS). The cell line used is SW620 and SW837, which are standard human colorectal adenocarcinoma cells. Tumours are then allowed to grow for three weeks until they become palpable and reach a diameter of 3-5 mm in the untreated control group. Tumour diameters are measured in two dimensions using a digital slide gauge. Tumour diameters are thereafter measured twice a week for the next 21 days. Each week blood is collected from each mouse by retroorbital bleeding under anaestaesia.

After 21 days all mice are euthanized, weight of tumours are determined, tumours are fixed and blood samples are taken.

Findings and Interpretation

The complexes or compositions of the invention dose-dependently inhibit tumor growth.

1. A chemical complex comprising:
i) a substituted or unsubstituted pyridine carboxy derivative according to formula 1 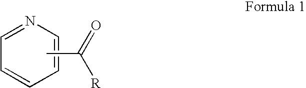
wherein R is selected from the group consisting of OH; OR′; NH₂; NHR′; NR′R″, O⁻Y⁺, and halogen, wherein R′ and R″ are independently selected from substituted and unsubstituted C₁—C₂₀ alkyl; and Y is a base addition salt of the free carboxylate; and
ii) an H2 histamine receptor antagonist.

2. A chemical complex according to claim 1, wherein the pyridine carboxy derivative of formula I is niacinamide.

3. A chemical complex according to claim 1, wherein the H2 histamine receptor antagonist is selected from the group consisting of ranitidine, cimetidine, famotidine, nizatidine and derivatives thereof.

4. A chemical complex according to any one of claims 1 to 3, wherein the molar ratio or weight ratio between the pyridine amide and the H2 histamine receptor antagonist is in the range of 1:10000 to 10000:1.

5. A chemical complex according to claim 4, further comprising a pharmaceutically acceptable carrier.

6. A pharmaceutical composition comprising:
i) a chemical complex as defined in any one of claims 1 to 3; and
ii) a pharmaceutically acceptable carrier.

7. The use of a chemical complex as defined in claim 1, or a composition comprising said complex for:
(a) the preparation of a medicament or a dietary supplement for immunomodulation in a mammal;
(b) for the preparation of a medicament or a dietary supplement for the suppression of hypersensitivity and/or inflammatory reaction in a mammal;
(c) for the preparation of a medicament for the treatment or prevention of atopic eczema, contact dermatitis, seborrhoeic eczema and/or psoriasis in a mammal;
(d) for the preparation of a medicament for the treatment or prevention of IgE mediated allergic reaction and/or condition in a mammal;
(e) for the preparation of a medicament for the alleviation of pain in a mammal; or
(f) for the preparation of a medicament or a dietary supplement for the treatment or prevention of cancer in a mammal.

8. The use according to claim 7, wherein the medicament or the dietary supplement is for the treatment or prevention of hypersensitivity skin disease in a mammal.

9. The use according to claim 7, wherein the medicament or the dietary supplement is for the treatment or prevention of asthma, allergic rhinitis, and/or anaphylaxis in a mammal.

10. The use according to claim 7, wherein the medicament or the dietary supplement is for the treatment or prevention of autoimmune disease and/or chronic inflammatory disease in a mammal.

11. The use according to claim 7, wherein the medicament or the dietary supplement is for the treatment or prevention of Crohn's disease, ulcerative colitis, rheumatoid arthritis, gout or osteoarthritis in a mammal.

12. The use according to claim 7, wherein the mammal is a human.

13. A method for immunomodulation in a mammal, which comprises administering a chemical complex as defined in any one of claims 1 to 3 or a composition comprising said complex to said mammal.

14. A method for the suppression of hypersensitivity and/or inflammatory reaction in a mammal, which comprises administering chemical complex as defined in any one of claims 1 to 3 or a composition comprising said complex to said mammal.

15. A method for the treatment or prevention of hypersensitivity skin disease in a mammal, which comprises administering a chemical complex as defined in any one of claims 1 to 3 or a composition comprising said complex to said mammal.

16. A method for the treatment or prevention of atopic eczema, contact dermatitis, seborrhoeic eczema and/or psoriasis in a mammal, which comprises administering a chemical complex as defined in any one of claims 1 to 3 or a composition comprising said complex to said mammal.

17. A method for the treatment or prevention of IgE mediated allergic reaction and/or condition in a mammal, which comprises administering a chemical complex as defined in any one of claims 1 to 3 or a composition comprising said complex to said mammal.

18. A method for the treatment or prevention of asthma, allergic rhinitis, and/or anaphylaxis in a mammal, which comprises administering a chemical complex as defined in any one of claims 1 to 3 or a composition comprising said complex to said mammal.

19. A method for the treatment or prevention of autoimmune disease and/or chronic inflammatory disease in a mammal, which comprises administering a chemical complex as defined in any one of claims 1 to 3 or a composition comprising said complex to said mammal.

20. A method for the treatment or prevention of Crohn's disease, ulcerative colitis, rheumatoid arthritis, gout or osteoarthritis in a mammal, which comprises administering a chemical complex as defined in any one of claims 1 to 3 or a composition comprising said complex to said mammal.

21. A method for the alleviation of pain in a mammal, which comprises administering a chemical complex as defined in any one of claims 1 to 3 or a composition comprising said complex to said mammal.

22. A method for the treatment or prevention of cancer in a mammal, which comprises a chemical complex as defined in any one of claims 1 to 3 or a composition comprising said complex to said mammal.

23. A method according to claim 13, wherein the mammal is a human.
